(19) United States
(12) Patent Application Publication
(10) Pub. No.: US 2021/0205805 A1
(43) Pub. Date: Jul. 8, 2021

(54) DEVICES AND METHODS FOR COLLECTING AND STORING FLUID SMAPLES FOR ANALYSIS

(71) Applicant: University of South Australia, Adelaide (AU)

(72) Inventors: Ricardo Jose FERREIRA NETO, Vilela (PT); Emily Frances HILDER, Millswood (AU); Andrew Aurthur GOOLEY, North Warrandyte (AU); Wei Boon HON, Croydon North (AU)

(21) Appl. No.: 17/059,674

(22) PCT Filed: May 30, 2019
(86) PCT No.: PCT/AU2019/000069

§ 371 Date: Nov. 30, 2020

(30) Foreign Application Priority Data
May 30, 2018 (AU) 2018901923

(51) Int. Cl. B01L 3/00; G01N 1/10; A61B 5/15

(52) U.S. Cl. CPC B01L 3/5023; G01N 1/10; A61B 5/150343; A61B 5/150351; A61B 5/150755; A61B 5/150022; G01N 2001/149; B01L 2300/069; B01L 2400/0406; B01L 2300/0832; B01L 2300/161; B01L 2300/04; B01L 2200/141; B01L 2300/0838

(57) ABSTRACT

A sampling device for collecting, storing and processing fluid samples for analysis is disclosed. The sampling device comprises a porous polymer monolith sampling substrate housed within a substantially impermeable housing. The housing surrounds the sampling substrate and further comprises a sampling aperture via which the sampling substrate is accessible externally from the sampling device.

### RELATED APPLICATIONS

The present application claims priority from PCT/AU2019/000069, filed May 30, 2019, and Australian Provisional Patent Application No. 2018901923 entitled “DEVICES AND METHODS FOR COLLECTING AND STORING FLUID SAMPLES FOR ANALYSIS,” filed May 30, 2018, the contents of which are hereby incorporated by reference in their entirety.

### TECHNICAL FIELD

The present disclosure relates to methods and devices for collecting, storing and processing samples for analysis. In a particular form, the present disclosure relates to methods and devices for collecting, storing and processing biological samples such as blood for analysis.

### BACKGROUND

There is a continuing need for devices and methods that can be used to analyse fluid samples to measure whether a specific analyte is present in a sample and/or how much of a specific analyte is present in a sample. Whilst these needs arise across a wide range of industries and pursuits, they are used extensively in the analysis of environmental fluid samples to assay for a particular analyte of interest such as an environmental contaminant, metal ion, essential mineral, organic material, etc. They are also used extensively in the analysis of biological fluid samples to assay for a particular analyte of interest such as a biomarker, biomolecule, etc.

In all of these cases, there is a need to collect and store the fluid sample or extract for subsequent analysis. Ideally, the fluid sample should be collected and stored using a device and method that is relatively easy to use for an unskilled user and that minimises the potential for contamination of the sample.

One example where fluid sample collection and storage is important is Dried Blood Spot (DBS) sampling. DBS sampling is a well-established protocol that involves collecting blood on a paper card and subsequently using the dried blood spots (DBS) for diagnostic purposes. DBS testing is predominantly used in the diagnosis of infectious diseases or the systematic screening of newborns for metabolic disorders. In more recent times, DBS testing has been investigated as a protocol for whole blood analysis. However, the use of DBS for the analysis of markers where interfering contaminations are detrimental is still limited. Even more recently, solid phase extraction (SPE) has been used in conjunction with DBS sampling in an effort to reduce the effects of interfering contaminations and improve signal to noise ratios. For example, in a process proposed by Spark Holland B.V., Emmen, Netherlands, (see www.sparkholland.com/?portfolio=dbs-autosampler) a cellulosic planar card is used for the initial DBS sampling and then the sample is “clamped”, to enable elution of the DBS sample which is then passed through an SPE cartridge as an enrichment step (see for example U.S. Pat. No. 8,586,382). However, this requires multi-step processing in order to reduce signal to noise ratios for analysis and does not address a need to minimise contamination of the sample during collection and storage.

United States Patent Application No. 20130116597A1 (Neoteryx) discloses the use of a device comprising a polymeric material for the collection of finger prick blood. United States Patent Application No. 20120276576A1 (Millispot) discloses a porous polymer material that has been developed for the collection of DBS samples adopting a planar format as an alternative to the paper-based cellulose materials currently being used. In each case, these polymer devices and sampling substrates fail to provide an effective solution to reduce sample contamination and/or improve signal to noise ratios.

There is thus a need to provide a device and/or a method for collecting, storing and processing fluid samples for analysis that reduces the risk of sample contamination during collection and storage. Alternatively, or in addition, there is a need to provide a device and/or a method for collecting, storing and processing fluid samples for analysis that improves signal to noise ratios in any subsequent analysis. Alternatively, or in addition, there is a need to provide a device and/or a method for collecting, storing and processing fluid samples that overcomes or ameliorates one or more of the problems associated with prior art methods and/or devices.

### SUMMARY OF THE INVENTION

In a first aspect, provided herein is a sampling device comprising a porous polymer monolith sampling substrate housed within a substantially impermeable housing, said housing surrounding the sampling substrate and further comprising a sampling aperture via which the sampling substrate is accessible externally from the sampling device.

In a second aspect, provided herein is an improved method of collecting and/or storing a sample for future analysis that minimises contamination of the sample, the method comprising:

providing a sampling device comprising a porous polymer monolith sampling substrate housed within a substantially impermeable housing, said housing surrounding the sampling substrate and further comprising a sampling aperture via which the sampling substrate is accessible externally from the sampling device;

collecting a fluid sample by contacting the sampling aperture directly or indirectly with a fluid under conditions for some of the fluid to transfer into the sampling substrate only through the sampling aperture; and

storing the sampling device with the sample sorbed into the sampling substrate for future analysis.

In practice, the inventors have found that analyses of fluid samples collected using the sampling device may have an improved signal to noise (S/N) ratio over known methods or devices, such as those described in, for example, U.S. Pat. No. 9,645,132. This improved S/N ratio may at least partially result from the nature of the material used for the sampling substrate and/or the way in which the sampling substrate is housed in the housing. The sampling substrate can be synthesised under controlled, optimised conditions and, in practice, this has been found to minimise background contaminants when compared with known sampling substrates that are prepared using natural materials, such as cellulose for example. The way in which the housing surrounds the sampling substrate also means that a user will naturally hold the sampling device by the housing when collecting a fluid sample and this then avoids contact between the user's fingers and the sampling substrate, thereby reducing possible contaminations.

In certain embodiments, the method further comprises at least partially drying the sample sorbed into the sampling substrate.

In some embodiments, the fluid sample is a bodily fluid. In these embodiments, the method can be used to collect and/or store samples of bodily fluids for future detection and/or measurement (i.e. analysis) of biological and/or environmental analytes in the bodily fluid.

In some particular embodiments, the fluid sample is blood or blood plasma. In these embodiments, the sampling device and method can be used in an improved Dried Blood Spot (DBS) collecting protocol. DBS is typically a paper-based technology collected by dripping blood onto a planar paper substrate. DBS sampling is commonly used to collect fluid samples for subsequent fatty acid analysis. Collection of DBS fluid samples has predominantly been assisted by a health professional and is hence not an intuitive process for self-collection. In contrast, the method described herein can be used to position the sampling device in contact with a drop of blood with minimal dexterity and/or minimal risk of contamination.

Thus, in a third aspect the present disclosure provides an improved method of collecting and/or storing a blood or blood plasma sample for future analysis that minimises contamination of the blood or plasma sample, the method comprising:

providing a sampling device comprising a porous polymer monolith sampling substrate housed within a substantially impermeable housing, said housing surrounding the sampling substrate and further comprising a sampling aperture via which the sampling substrate is accessible to a fluid externally from the sampling device;

providing a blood sample;

collecting a sample of the blood by contacting the sampling aperture directly or indirectly with the blood under conditions for some of the blood to transfer into the sampling substrate only through the sampling aperture;

at least partially drying the sample of blood on the sampling substrate; and

storing the sampling device with the blood sample sorbed into the sampling substrate for future analysis.

In some particular embodiments, the fluid is a sample for future analysis for metals, metal ions or essential minerals. The fluid sample in these embodiments may be an aqueous sample, a bodily fluid, an environmental sample, etc.

Thus, in a fourth aspect the present disclosure provides an improved method of collecting and/or storing a sample for future analysis for the presence and/or amount of one or more metals, metal ions or essential minerals that minimises contamination of the sample, the method comprising:

providing a sampling device comprising a porous polymer monolith sampling substrate housed within a substantially impermeable housing, said housing surrounding the sampling substrate and further comprising a sampling aperture via which the sampling substrate is accessible to a fluid externally from the sampling device;

providing a fluid sample to be analysed for one or more metals, metal ions or essential minerals;

collecting a fluid sample by contacting the sampling aperture directly or indirectly with the fluid under conditions for some of the fluid to transfer into the sampling substrate only through the sampling aperture;

optionally, at least partially drying the sample of fluid on the sampling substrate;

storing the sampling device with the fluid sample sorbed into the sampling substrate for future analysis; and

determining the metal, metal ion or essential mineral composition of the fluid sample sorbed to the sampling substrate.

In some embodiments, the method of the second, third or fourth aspects further comprises eluting the sorbed sample from the sampling substrate and analysing the amount of one or more target analytes in the eluted sample.

In some embodiments, the sampling device is configured for use in an instrument for subsequent extraction and analysis, such as an SPE instrument.

In some embodiments which are used for blood sampling, the sampling device can be configured for use in one of a range of blood sampling systems or protocols, including but not limited to hemaPEN (Trajan), Neoteryx (Mitra), OHSU (Touch Spot), hemaXis (DBS System), AutoCollect (Ahlstrom), HemoLink (Tasso, Inc.), Capitainer (Capitainer), TAP100 Touch Activated Phlebotomy (7th Sense Bio), HemaSpot HF (Spotonsciences), PTS Pod™ Blood Collection System (PTS Diagnostics), and Fluispotter (Fluisense).

Thus, in a fifth aspect, provided herein is a method for determining an amount of a target analyte in a fluid sample, the method comprising:

providing a sampling device comprising a porous polymer monolith sampling substrate housed within a substantially impermeable housing, said housing surrounding the sampling substrate and further comprising a sampling aperture via which the sampling substrate is accessible externally from the sampling device;

collecting a fluid sample by contacting the sampling aperture directly or indirectly with a fluid under conditions for some of the fluid to transfer into the sampling substrate only through the sampling aperture;

storing the sampling device with the sample sorbed into the sampling substrate;

eluting the sorbed sample from the sampling substrate; and

determining the amount of the target analyte in the eluted sample.

In certain embodiments, the sampling substrate comprises a porous polymer and a hydrophilic coating on the porous polymer. The hydrophilic coating assists with wicking of the fluid sample into the sampling substrate. This then allows for the sampling device to be used without a user's fingers contacting the fluid sample, thereby further reducing actual or potential contamination of the sample prior to or during sample collection.

In certain embodiments, the sampling device comprises a removable seal or cap covering the sampling aperture and the method comprises removing the removable seal or cap immediately prior to collecting the sample. In this way, the sampling device can be manufactured or prepared in a controlled ‘clean’ environment and sealed or capped using the removable seal or cap in that environment. This prevents or reduces the risk of contamination of the sampling substrate during transport and/or storage or before use.

In certain embodiments, the sampling device comprises a removable cap, and the cap further comprises a blood collection capillary tube of a predetermined volume. Using the blood collection capillary tube in the cap, an accurate blood volume can be collected from a site of puncture (e.g. a finger, heel or ear lobe) and the capillary tube and the sampling substrate are then brought into contact with one another to initiate blood transfer from the capillary tube onto the sampling substrate. This further prevents or reduces the risk of contamination of the sampling substrate as well as collecting a predetermined volume of fluid. The blood collection capillary tube can be of any of the designs known to those skilled in the art and can be coated with an anti-coagulant such as heparin or EDTA.

In some particular embodiments, the fluid sample is blood or blood plasma and the target analyte(s) are one or more fatty acids. This, in a sixth aspect, provided herein is a method for determining the fatty acid composition of a fluid sample comprising fatty acids, the method comprising:

providing a sampling device comprising a porous polymer monolith sampling substrate housed within a substantially impermeable housing, said housing surrounding the sampling substrate and further comprising a sampling aperture via which the sampling substrate is accessible externally from the sampling device;

collecting a fluid sample by contacting the sampling aperture directly or indirectly with a fluid sample under conditions for some of the fluid to transfer into the sampling substrate only through the sampling aperture;

storing the sampling device with the sample sorbed into the sampling substrate; and

determining the fatty acid composition of the sample sorbed to the sampling substrate.

The fatty acid composition may be determined by methods known to those skilled in the art, for example by derivatisation of the fatty acids in the sorbed sample and analysis of the resulting derivatised compounds by gas chromatography (GC).

In a seventh aspect, provided herein is a kit for collecting and storing a blood sample from a subject, the kit comprising:

a sampling device comprising a porous polymer monolith sampling substrate housed within a substantially impermeable housing, said housing surrounding the sampling substrate and further comprising a sampling aperture via which the sampling substrate is accessible externally from the sampling device;

a sharp object for obtaining a blood sample from the subject; and

instructions for use.

### BRIEF DESCRIPTION OF DRAWINGS

Embodiments of the present invention will be discussed with reference to the accompanying figures, which are examples of the scope of the invention and do not limit the scope of the invention, wherein:

FIG. 1 is an exemplary view of a porous polymer sampling substrate of the invention;

FIG. 2 is an exemplary view of a tube as an impermeable housing of the invention;

FIG. 3 is an exemplary view of the porous polymer sampling substrate of FIG. 1 within a section of the tube of FIG. 2 being prepared for blood collection; and FIG. 3A is an exemplary view showing blood collection using the sampling device shown in FIG. 2;

FIG. 4 is an exemplary view showing blood collection using the sampling device shown in FIG. 2;

FIG. 5 is an exemplary view of the sampling device shown in FIG. 2 with the cap on the device after sample collection;

FIG. 6 is a photograph of an alternative embodiment of a sampling device of the present disclosure integrated with a hemaPEN® device;

FIG. 7 shows an alternative embodiment of a sampling device of the present invention wherein the fluid sample is collected via a capillary tube with minimal exposure area to the elements during blood collection;

FIG. 8 is an exemplary view of a sampling device shown in FIG. 7;

FIG. 9 are close-up views of part of the sampling device depicted in FIG. 7 before use (upper photograph) and after fluid sample collection (lower photograph);

FIG. 10 is a plot showing the assayed background ratio for various metals between a blank DBS paper and the background of the methacrylate polymers (Black) and the divinylbenzene polymer (Grey) (Plotted ratio=background found on Perkin Elmer PKI 226 divided by the background found on the polymers prepared and disclosed); and

FIG. 11 is a plot showing the influence of the background contaminations in the percentage of recovery reported after subtracting background signal. (Plotted value=% of recovery with background−% of recovery after background subtraction). (Left Black=DVB sampling substrate of the present disclosure; Left Grey=methacrylate sampling substrate of the present disclosure; Right Black=commercial DBS paper substrate (Whatman® 903 protein saver card); Right Grey=commercial DBS paper substrate (Perkin Elmer PM 226)).

In the following description, like reference characters designate like or corresponding parts throughout the figures.

### DESCRIPTION OF EMBODIMENTS

As used herein, the term “about” refers to a range of numbers that a person of skill in the art would consider equivalent to the recited value in the context of achieving the same function or result.

As used herein, the terms “a” and “an” refer to one or to more than one (i.e. to at least one) of the grammatical object of the article. By way of example, “an element” means one element or more than one element.

Disclosed herein are improved sampling devices and methods of collecting and/or storing a fluid sample for future analysis that minimises contamination of the fluid sample. As discussed, development and utilisation of known methods for DBS based analysis of markers remain limited due to the poor signal to noise (S/N) ratios attained post sample extraction.

The sampling device 10 disclosed herein comprises a porous polymer monolith sampling substrate 12 housed within a substantially impermeable housing 14. The housing 14 surrounding the sampling substrate 12 further comprises a sampling aperture 16 via which the sampling substrate 12 is accessible externally from the sampling device 10.

The method disclosed herein comprises providing a sampling device 10 comprising a porous polymer monolith sampling substrate 12 housed within a substantially impermeable housing 14. The housing 14 surrounds the sampling substrate 12 and further comprises a sampling aperture 16. In use, the sampling substrate 12 is only accessible externally from the sampling device 10 via the sampling aperture 16.

In the method, a fluid sample 20 is collected by contacting the sampling aperture 16 with a fluid under conditions for some of the fluid to transfer into the sampling substrate 12 only through the sampling aperture 16. The sampling aperture 16 can be contacted directly with the fluid or indirectly with the fluid by transferring the fluid to the sampling aperture 16 using a collector such as a capillary tube in fluid connection with the sampling aperture 16. After collection, the sampling device 10 is stored with the sample sorbed into the sampling substrate 12 for future analysis.

An advantageous embodiment of the sampling device 10 is shown in FIGS. 2 to 5 in which the housing 14 is in the form of a cylindrical solid phase extraction (SPE) cartridge having the sampling substrate 12 positioned adjacent a tip of the cartridge. The SPE cartridge may have a volume of 3 mL, 6 mL, 12 mL, 20 mL or 60 mL. One solution to improve S/N ratios in the future analysis of the sample sorbed on the sampling substrate 12 is through the use of SPE. Sampling devices 10 having the configuration shown in FIGS. 2 to 5 can advantageously be integrated for automated workflow extraction.

The methods and devices of the present disclosure can be used to collect and store a wide range of samples. As discussed, the sampling of blood by DBS is widely practiced and is a common method for collecting and storing blood samples for future fatty acid (FA) analysis. The methods and devices of the present disclosure can be used for any of the fluid sample collection and storage protocols for which DBS is used currently and in the future. In addition, the methods and devices of the present disclosure can also be used to collect and store non-blood samples and non-biological samples, particularly water-based or aqueous samples. For example, the methods and devices of the present disclosure can be used to collect and store environmental samples for future analysis for analytes of interest, such as metals, metal ions, essential minerals, organic material, biological material, hydrocarbons, or any other environmental contaminant.

In certain embodiments, the fluid sample to be collected and stored is a biological sample. The biological sample may be a bodily fluid, for example, blood, saliva, breast milk, urine, semen, blood plasma, synovial fluid, serum and the like.

The analyte of interest in the bodily fluid may be a biomolecule present in the bodily fluid or suspected of being present in the bodily fluid. The biomolecule may be any protein, peptide or amino acid, including unlabelled or labelled antibodies, receptors, hormones, growth factors and modified proteins, nucleic acids, proteins and peptides of infectious origin; any nucleic acid like DNA or RNA; any nucleotide, oligonucleotide or polynucleotide; PNAs (peptide nucleic acids); any metabolite; any lipid; any fatty acid; sugar (monomer, oligomer or polymer); proteoglucans; any low molecular pathway product, signal molecule, receptor or enzyme activator or inhibitor; agents, medicaments and metabolites of medicaments, medicaments or any other biomolecule of interest.

In other certain embodiments, the fluid sample may be an oil comprising fatty acids (for example fish oil, cooking oil, seed oil, food supplements, nutritional supplements, etc).

Experiments conducted by or on behalf of the inventors have shown that the methods and devices of the present disclosure minimise contamination of the sample. For example, the inventors' results have been compared against those described by U.S. Pat. No. 9,645,132B2 (Gibson et al.) and it was concluded that fluid samples collected and stored using the methods and devices of the present disclosure have, overall, an improved S/N ratio. Moreover, the substrate used by Gibson et al. is not hydrophilic enough to wick blood (against gravity) and traditional cellulosic DBS paper was used as the substrate.

As used herein and in the context of the present specification, the term “contaminant”, and related or similar terms, means a material or substance that, if present on or in the sampling substrate, would increase or decrease the assayed amount of an analyte present in the sample sorbed on the sampling substrate, as compared to the amount of the analyte present in the sample prior to application to the sampling substrate.

The sampling device 10 can take any suitable form. In advantageous embodiments, the sampling device 10 is in a form or is configured to allow it to be used in any commercially available assay procedure, protocol, device, machine or instrument. By way of example, a wide range of commercial protocols and instruments are available for assaying biological molecules of interest in blood samples. These include hemaPEN (Trajan), Neoteryx (Mitra), OHSU (Touch Spot), hemaXis (DBS System), AutoCollect (Ahlstrom), HemoLink (Tasso, Inc.), Capitainer (Capitainer), TAP100 Touch Activated Phlebotomy (7th Sense Bio), HemaSpot HF (Spotonsciences), PTS PodTM Blood Collection System (PTS Diagnostics), and Fluispotter (Fluisense). The sampling device 10 disclosed herein can be integrated into or form part of any of the sampling devices used with these protocols. For example, the sampling substrate 12 can be included in hemaPEN (Trajan) as shown in FIG. 6. In these embodiments, a capillary tube of the hemaPEN is used to draw in a blood sample and transfer it to the sampling substrate 12. It will be appreciated that in these embodiments, the capillary tube functions as the sampling aperture 16. It will also be appreciated that in these embodiments, the capillary tube functions to transfer a predetermined volume of fluid sample 20 to the sampling substrate 12. Sampling devices of these embodiments are particularly suitable for use in “one-step” easy extraction and automation protocols.

The sampling substrate 12 is a porous polymer monolith (PPM). Advantageously, the porous polymer monolith is prepared in a controlled environment and this minimises the presence of background contaminants in the sampling substrate 12. This then means that the sampling device 10 can be used for the analysis of ubiquitous compounds by significantly reducing background contamination levels. In particular, the present inventors postulate that contaminants present in cellulose-based sampling or DBS devices can interfere with the accurate determination of the amount of a particular analyte of interest.

In certain embodiments, the PPM sampling substrate 12 comprises less than about 1 μg/cm² of contaminants, such as less than about 0.5 μg/cm² of contaminants.

The PPM sampling substrate 12 is formed from any polymeric material that provides a suitable porosity. The porous polymer monolith may be formed by polymerisation of one or more monomers in the presence of two or more porogens. The porogens may be a selected ratio of porogenic solvents. Suitable porogenic solvents, or porogens, may typically be a mixture of one or more alcohols and one or more alkanes. A useful mixture of alcohols and alkanes may include methanol, dodecanol, n-hexane, and cyclohexanol. For example, the PPM sampling substrate 12 may be formed using any of the methods disclosed in international patent publication WO 2011/082449, international patent publication WO 2013/006904 or international patent publication WO 2017/088032. Polymeric divinylbenzene (DVB) and polymeric methacrylate materials are particularly suitable. The PPM sampling substrate 12 may comprise at least 50% (w/w), at least 60% (w/w), at least 70% (w/w), at least 80% (w/w) at least 90% (w/w), at least 95% (w/w) or at least 99% (w/w) of the desired polymeric material.

The PPM sampling substrate 12 can be fabricated in situ in a tubular body by electromagnetic radiation, e.g. ultraviolet, initiation. For this purpose, the cross-linking initiator is an appropriate radiation responsive initiator known to those skilled in the art. A suitable reagent for ultraviolet initiation is 2,2-dimethoxy-2-phenylacetone (DMPA), phenylbis (2,4,6-trimethylbenzoyl)-phosphine oxide (BAPO), or any other UV initiator known to the person skilled in the art.

Synthesis of the PPM sampling substrate 12 can be used to form sampling substrates of any suitable dimension, such as between 0.05 mm and 0.005 mm, or between 1 mm and 0.05 mm, or between 10 mm and 1 mm, or between 50 mm and 10 mm.

In use, the fluid sample 20 is sorbed into the PPM sampling substrate 12. As used herein, the term “sorbed” means that the fluid sample 20 is bound, absorbed, adsorbed or chelated to the sampling substrate 12.

If desired, the PPM sampling substrate 12 may further comprise additional material, such as any inert material like e.g. agarose, Sephacryl resin, silicone, latex, polysaccharides, cellulose ether, and derivatives, thermosetting of thermoplastic polymers, metals, particles, etc. in addition to the polymeric material.

In certain embodiments, the sampling substrate 12 further comprises a hydrophilic coating on the porous polymer. Alternatively, or in addition, the PPM sampling substrate 12 may be formed by copolymerisation with a hydrophilic monomer, such as 2-hydroxyethylmethacrylate (HEMA). The hydrophilic coating assists with wicking of the fluid sample 20 into the sampling substrate 12 and, for example, blood is able to be collected through a capillary force wicking membrane. This then allows for the sampling device 10 to be used without a user's fingers contacting the sample, thereby further reducing actual or potential contamination of the sample prior to or during sample collection. By way of example, the inventors' studies have shown that a porous polymer material coated with 5% of a hydrophilic coating wicked a defined amount of blood against gravity faster than a commercially available PUFAcoat paper (a derivate of Whatman SG81 ion exchange paper which is a composite of cellulose and large pore silica) which was not able to wick against gravity and faster than a traditional cellulosic substrate.

Any coating material that is known in the art to increase the wettability of a surface or any hydrophilic coating material that is able to coat the porous polymer can be used in the hydrophilic coating. Suitable coating materials include, but are not limited to polyethylene glycol (PEG), polyvinyl alcohol (PVA), polyvinylpyrrolidone (PVP), polyacrylic acid (PAA), polyacrylic maleic acid (PAMA), and poly(ethylene glycol)methyl ether methacrylate (PEGMA). In certain embodiments, the coating comprises PEGMA.

Alternatively, or in addition, a coating may be used to reduce the number of unspecific binding interactions. Such coatings include detergent blockers such as Tween-20 and Triton X-100; protein blockers such as bovine serum albumin, casein, fish gelatin, and whole sera; and polymer-based blockers such as polyethylene glycol (PEG), polyvinyl alcohol (PVA), polyvinylpyrrolidone (PVP), polyacrylic acid (PAA), and polyacrylic maleic acid (PAMA).

The coating(s) may be present on the porous polymer in an amount of from about 1% (w/w) to about 10% (w/w), such as about 1% (w/w), about 2% (w/w), about 3% (w/w), about 4% (w/w), about 5% (w/w), about 6% (w/w), about 7% (w/w), about 8% (w/w), about 9% (w/w) or about 10% (w/w). In certain embodiments, the coating(s) is/are present on the porous polymer in an amount of about 5% (w/w).

In certain embodiments, the sampling substrate 12 is also coated with an anti-oxidant. The coating may be applied by contacting the sampling substrate 12 with a solution containing an anti-oxidant and drying. Suitable anti-oxidants include, but are not limited to resveratrol, t-butylhydroquinone, BHT, BHA, citric acid, citrate, ascorbic acid, ascorbate, flavanoids such as bacalein, and antioxidant plant extracts. The anti-oxidant(s) may be present on the sampling substrate 12 in an amount between about 0.001 mg and about 10 mg, or in an amount between about 0.01 mg and about 1 mg, or in an amount between about 0.01 mg and about 0.5 mg.

The sampling substrate 12 can be any shape, such as circular, rectangular, square, etc.

The sampling substrate 12 is housed in a substantially impermeable housing 14. The substantially impermeable housing 14 can be formed from any material that prevents or reduces the transfer of the fluid and/or the analyte of interest therethrough. Suitable materials include plastic, metal, glass, porcelain or similar. Thermosetting or thermoplastic resins like polypropylene, polyethylene, polypropylene-copolymers, polyvinylchloride, polyurethane, polycarbonate, polyamide, polyimide, polystyrene, polyethyleneterephthalate, polylactide, ethylene-polyvinylacetate, vinylchloride vinylacetate copolymers, polyacetals, polyetheralcohols, vinylacetate copolymers or acrylic polymers are particularly suitable.

The substantially impermeable housing 14 encloses the sampling substrate 12 and further comprises a sampling aperture 16 through which the fluid sample 20 is able to contact the sampling substrate 12. The sampling substrate 12 is only accessible externally from the sampling device 10 via the sampling aperture 16. This means that a user will naturally hold the sampling device 10 by the housing 14 when collecting a fluid sample 20 and this then avoids contact between the user's fingers and the sampling substrate 12, thereby reducing possible contaminations.

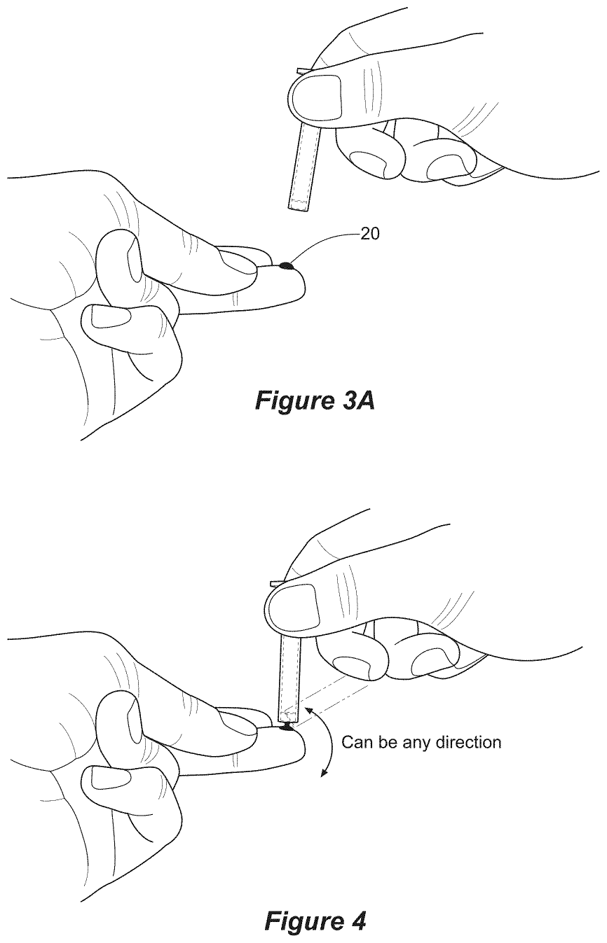

The sampling aperture 16 can be positioned on any suitable surface of the substantially impermeable housing 14. Typically, the sampling aperture 16 is positioned on a surface of the substantially impermeable housing 14 that will be brought in to contact with the fluid sample 20 in normal use. In the embodiment that is illustrated in FIGS. 3 and 4, the sampling aperture 16 is positioned on the tip or end surface of the cartridge housing 14.

The sampling aperture 16 can be any shape, such as circular, square, ellipsoid, triangular, etc. The size of the sampling aperture 16 may be from about 10 μm to about 50 mm in diameter in the case of a circular sampling aperture 16. In certain embodiments, the size of the sampling aperture 16 is from about 1 to about 13 mm in diameter, such as about 2 to about 5 mm in diameter. In the illustrated embodiments, the size of the sampling aperture 16 is 3.6 mm in diameter.

In certain embodiments, the sampling device 10 comprises a removable seal or cap 18 covering the sampling aperture 16. The removable seal or cap 18 is removed immediately prior to obtaining the fluid sample 20. In this way, the sampling device 10 can be manufactured or prepared in a controlled ‘clean’ environment and sealed or capped using the removable seal or cap 18 in that environment. This prevents or reduces the risk of contamination of the sampling substrate 12 during transport and/or storage or before use. The removable seal or cap 18 can also be reattached to the sampling device 10 after the fluid sample 20 has been collected.

In use, a fluid sample 20 is collected by contacting the sampling aperture 16 with the fluid under conditions for some of the fluid to transfer into the sampling substrate 12 only through the sampling aperture 16. As discussed earlier, the hydrophilic coating on the sampling substrate 12 assists in wicking blood and other fluid samples into the sampling substrate 12. This then means that the sampling device 10 can be applied to the fluid to be sampled at various angles and the fluid sample 20 will still ‘wick’ into the sampling substrate 12 through the sampling aperture 16. This enables the direct collection of blood samples in a seamless and user-friendly way.

The fluid sample 20 may be applied to the sampling substrate 12 in an amount that is less than about 100 μL, or less than about 90 μL, or less than about 80 μL, or less than about 70 μL, or less than about 60 μL, or less than about 50 μL, or less than about 40 μL, or less than about 30 μL, or less than about 25 μL, or less than about 20 μL, or less than about 15 μL, or less than about 10 μL, such as about 5 μL. Advantageously, the dimensions of the sampling substrate 12 can be used to control the volume of fluid sample 20 that transfers into the sampling device 10. Alternatively, or in addition, a capillary tube such as those found on a hemaPEN, can be used to apply a volumetric dose of the fluid sample 20 to the sampling substrate 12. It will be appreciated from the foregoing that the devices and methods disclosed herein are suitable for use in volumetric absorptive microsampling (YAMS) procedures.

It will be appreciated that the devices and methods disclosed herein are suitable for obtaining fluid samples 20 for microsampling. Microsampling involves capturing and analysing minute (e.g. 10-20 μL) fluid samples 20 for analysis. Reduced sample sizes make sample collection easier for patients and clinicians. However, reduced sample sizes also make analysis more difficult and/or problematic because background or external contamination has a more significant impact on the analysis than with larger sample volumes (e.g. samples of up to 10 mL obtained by venepuncture). Therefore, contamination by the sampling substrate 12 and/or external sources is a major issue in microsampling collection and analysis procedures.

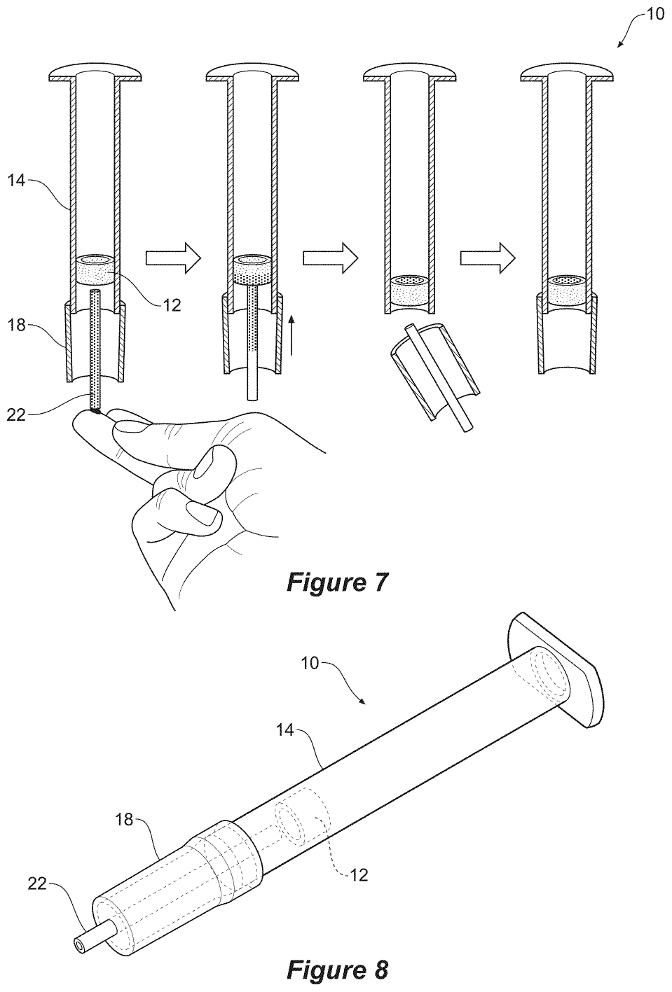

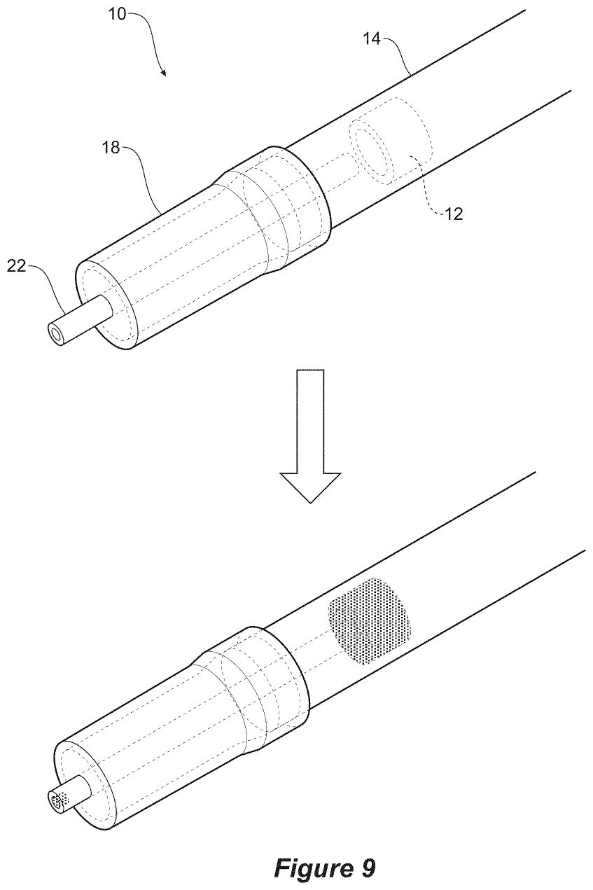

A further embodiment of the sampling device 10 is shown in FIGS. 7 to 9, which shows a sampling device 10 comprising a removable cap 18, and the removable cap 18 further comprises a blood collection capillary tube 22 of a predetermined volume. Using the blood collection capillary tube 22 in the removable cap 18, an accurate sample volume can be collected from a site of puncture (e.g. a finger, heel or ear lobe). After collection, the capillary tube 22 and the sampling substrate 12 are then brought into contact with one another to initiate blood transfer from the capillary tube 22 onto the sampling substrate 12. The capillary tube 22 and the sampling substrate 12 can be brought into contact with one another by the user pressing against the tip of the capillary tube 22 at the time of fluid sample 20 collection. After the fluid sample 20 has been transferred from the capillary tube 22 to the sampling substrate 12 a new removable cap 18 can be attached to the sampling device 10. This embodiment further prevents or reduces the risk of contamination of the sampling substrate 12 as well as collecting a predetermined volume of fluid sample 20. The capillary tube 22 can be of any of the designs known to those skilled in the art and when it is used for blood collection it can be coated with an anti-coagulant such as heparin or EDTA.

The internal diameter of the capillary tube 22 (and hence the effective diameter of the sampling aperture 16) may be from about 10 μm to about 3 mm, such as from about 0.3 mm to about 2 mm in diameter. The internal diameter of the capillary tube 22 shown in the illustrated embodiments is 0.95 mm and the capillary tube 22 is 28.2 mm in length.

After collection, the sampling device 10 is stored with the fluid sample 20 absorbed into the sampling substrate 12 for future analysis. The analysis may be performed weeks or months after sample collection.

The stored sample may be analysed using any suitable analysis technique known in the art. For example, the sample may be extracted from the sampling device 10 using standard SPE techniques and devices and the eluate analysed by MS, GC-MS, HPLC, HPLC-MS, etc.

The features and benefits of the sampling device 10 and methods disclosed herein are:
PPM sampling substrate 12 is prepared in a controlled environment providing a less contaminated sampling substrate 12 and improving signal to noise ratio for low abundant biomarkers/analytes particularly in exposure science;
A highly wicking PPM sampling substrate 12 gives the capability of wicking against gravity (fluid sample 20 collection in any direction);
Dried Blood Spot is typically a paper-based technology collected by dripping blood onto a planar paper substrate. The collection has predominantly been assisted by a health professional and is hence not an intuitive process for self-collection. In contrast, the sampling device 10 disclosed herein can be adapted into any embodiment, for example, an SPE cartridge, and can be positioned with minimal dexterity to collect the blood from any source. The cartridge embodiment of the DBS is well suited to laboratory workflows and can be easily positioned into an SPE instrument for extraction and analysis; and
The PPM sampling substrate 12 can be easily be adapted into any embodiment including but not limited to hemaPEN (Trajan); Neoteryx (Mitra); OHSU (Touch Spot); hemaXis (DBS System).

The methods described herein may be used by nutritionists, the general population with increased awareness towards prevention of diseases; environmental scientists; governments with a desire to implement healthier preventative measures, etc. The methods can be used by health professionals and consumers for personal home testing for dietary and wellbeing purposes.

Future applications are intended to provide a registered test kit for health professionals and consumers for personal home testing for dietary and wellbeing purposes.

### EXAMPLES

Specific embodiments of the sampling device and method of the present disclosure are described in the following non-limiting examples.

### Example 1—Production of the Sampling Device

Sampling substrates were prepared as porous polymer monoliths (PPM) through UV initiated polymerisation of methyl methacrylate, the hydrophilic functional monomer 2-hydroxyethyl methacrylate (HEMA), the crosslinking monomer ethylene glycol dimethacrylate (EGDMA), porogens methanol and hexane using the photoinitiator phenylbis (2,4,6-trimethylbenzoyl)-phosphine oxide (BAPO) in 3.6 mm I.D. polyethylene tubing. The polymer material was cast inside a polyethylene tube with 5.6 mm O.D.×3.6 mm I.D.×120 mm length. After polymerization, small discs of a nominal 3.5 mm length were prepared and washed using Soxhlet extraction. The sampling substrates were coated with poly(ethylene glycol)methyl ether methacrylate (PEGMA) to increase their blood absorption properties.

Sampling substrates of the present disclosure and commercial DBS paper substrates (PM 226) were coated with an anti-oxidant solution and air dried. Stability of the antioxidant used was evaluated over a period of 6 months, and it was found that the antioxidant was active and above the required concentration to still be effective.

### Example 2—Fatty Acid Analysis of Blood Samples

Donor blood was first collected into EDTA coated tubes and pipetted onto the sampling substrates.

Finally, the PPM sampling substrate material was introduced into a 1 mL SPE cartridge housing that allows an easy fluid sample collection and sample dispensing (FIG. 2). The sampling substrate was protected with an LDPE cap.

In use, the sampling device is prepared by removing the cap. A finger prick is then done according to Centers for Disease Control and Prevention procedure (https://www.cdc.gov/labstandards/pdf/vitaleqa/poster_capillaryblood.pdf) to provide a blood droplet. The sampling device is then brought into contact with the blood droplet by applying the tip of the sampling device to the surface of the blood droplet (can be any direction) (FIG. 4). The PPM sampling device was proven to be efficient in collecting fluid samples with different hematocrit levels, up to 95%.

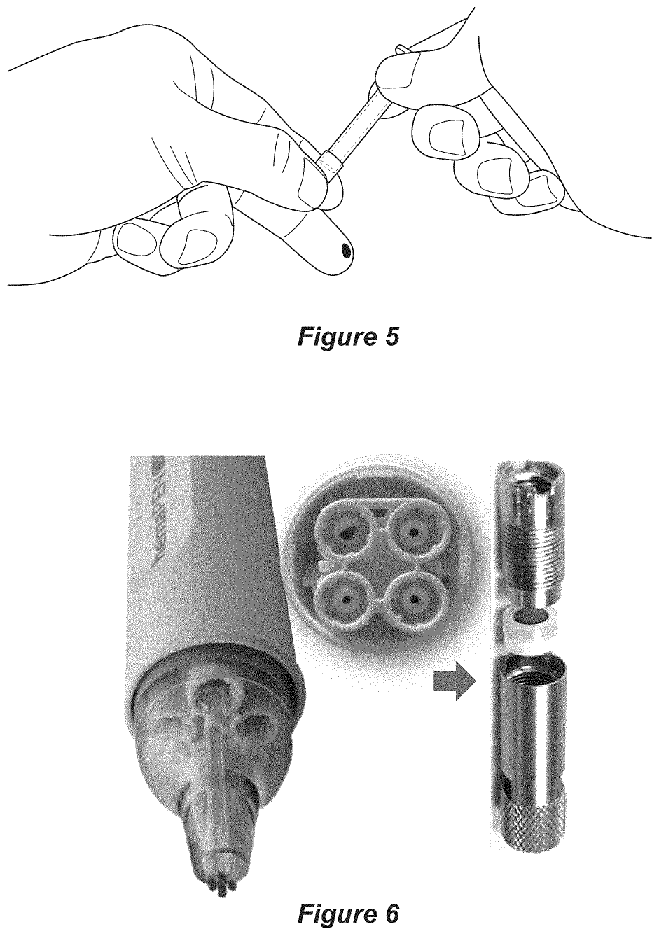

Once the fluid sample is collected, the cap is placed onto the device. The device is then placed in a polyfoil bag with desiccant to dry overnight. The polymer can be removed with a pushing jig for analysis (FIG. 5).

The performance of the sampling device comprising the PPM sampling substrate was compared to a commercially available DBS substrate (PUFAcoat) and to a standard DBS paper (PM 226) for the analysis of fatty acids (FA) by GC-MS.

Four different elements were evaluated to fully characterize the polymeric material as a viable option for the analysis of FA.

The PPM sampling substrate was shown to be the one that wicked a defined amount of blood against gravity faster when coated with 5% of the hydrophilic PEGMA coating (5.46±0.4 s). The commercially available DBS paper for the analysis of FA was not able to wick against gravity and a traditional cellulosic substrate wicked the blood in 14.4±1.4 s.

The PPM sampling substrate (due to its synthetic nature) was prepared in a well-controlled environment and showed less background contamination. This is important when using smaller amounts of blood (5 μL) where the difference between accounting or not accounting for the contaminations introduces a 0.15% difference in the final result in terms of total difference to the whole blood sample. Using the same volume of blood, this difference is 0.063% for the commercially available paper for FA analysis and 1.5% for the traditional DBS paper.

In terms of extraction efficiency, the PPM sampling substrate systemically led to smaller differences to the blood control in terms of overall differences when compared to the other materials tested (PPM 2.9±0.4%; PUFAcoat 3.48±0.02; PM 226 6.2±0.5% when using 5 μL of blood).

The study was conducted over a period of 28 days and the FA stability was evaluated overall and by FA class. Overall, there were no significant differences between the PPM sampling substrate and the PUFAcoat material. The degradation of some classes of FA was noticeable when using the PM 226 substrate over time.

### Example 3—Analysis of Heavy Metals and Essential Minerals

The sampling device was used for the analysis of other ubiquitous elements such as heavy metals and minerals. It was found that the commonly used DBS substrates have more contaminations that may interfere with the analysis of these analytes. This was particularly pronounced when analysing for elements such as Mn, Ca, Na, Mg, Fe, all of which are commonly used for diagnostic purposes. Nevertheless, it was found that this was also the case for other elements with diagnostic relevance, such as Pb, As and Cd.

Two experiments were conducted—one was conducted to assess the amount of contamination of the diverse sampling substrates used and the other was conducted to quantify the impact of any background contamination on the reported results.

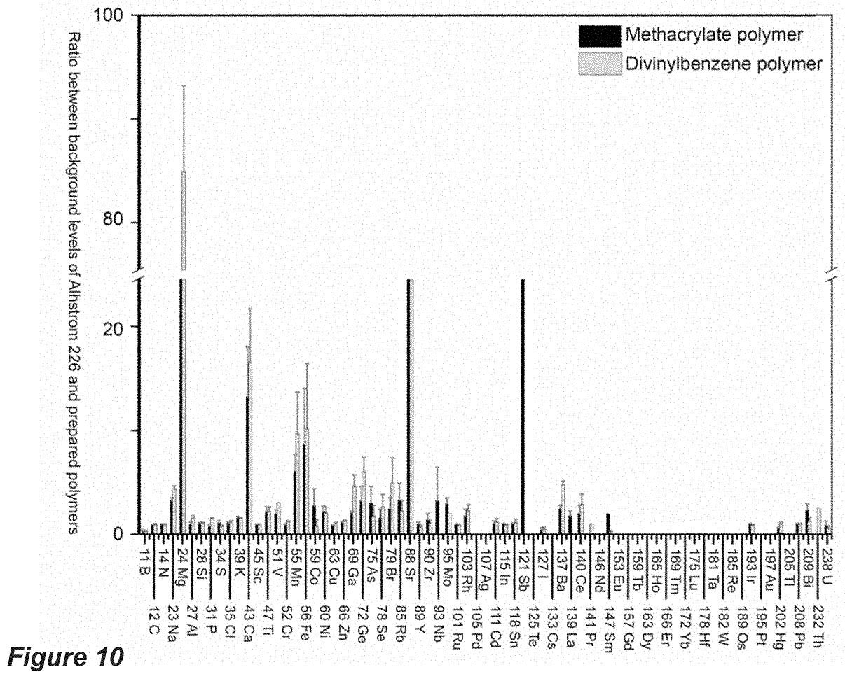

One of the experiments comprised the analysis of the background levels by extraction with 5% acetic acid in the presence of 0.01% of Triton X100. This study was semi-quantitative with the purpose of demonstrating that the presence of several heavy metals and minerals was more pronounced in the commonly used DBS substrate (in this case PKI 226). Several 0.8 mm² pieces of PM 226 paper were cut (an area roughly necessary to absorb 20 μL of bodily fluids), placed inside plastic tubes (acid pre-washed), and the extraction performed with constant agitation (300 rpm) at room temperature. A similar extraction process was carried out using the prepared PPM substrates inside a 3.5 mm I.D. housing and with 3.5 mm length. The results are summarised and represented in FIG. 10. The results in FIG. 10 show that for some elements the background was higher in the DBS paper compared with the background found in the sampling substrate.

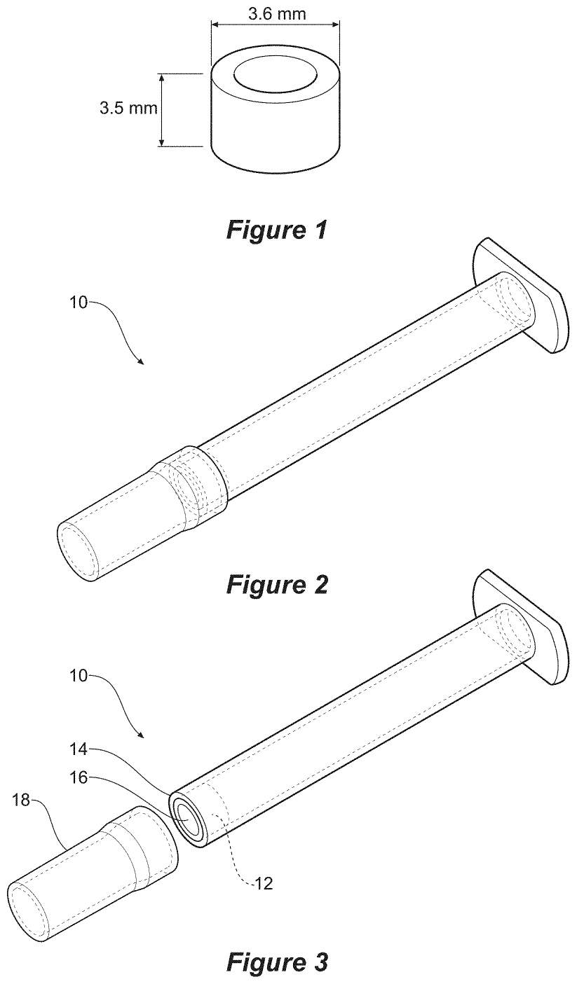

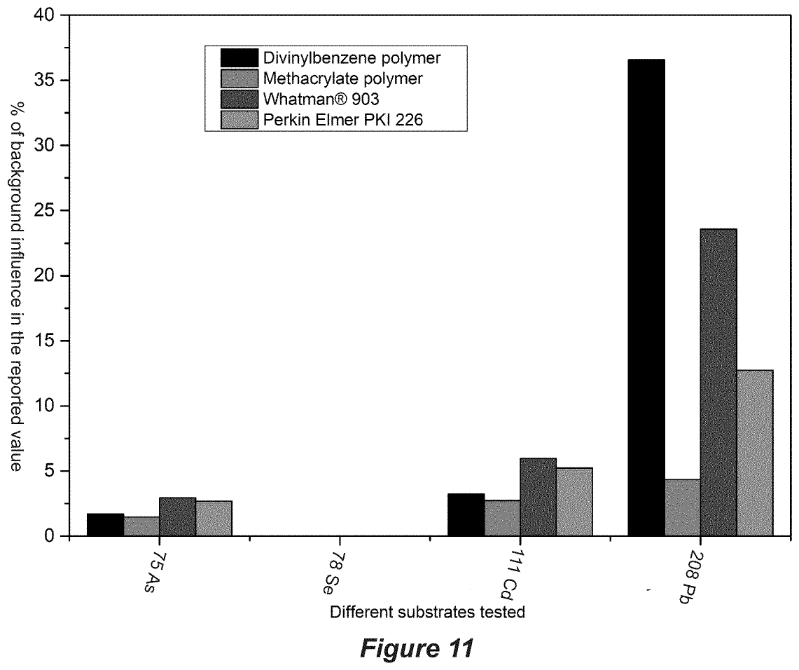

The second experiment was designed to assess the impact of background contaminations in the final results when looking at specific metals, particularly, As, Se, Cd, and Pb. Animal blood was used in this experiment. A 20 μL drop of blood was placed on the PPM prepared for this purpose in the embodiment described in FIG. 1. In parallel, 20 μL of blood was placed on two commonly used DBS substrates, namely PKI 226 and Whatman® 903. Extraction was performed in 1.5 mL of 5% HNO₃ in the presence of 0.01% Triton X 100, inside a pre-washed plastic tube with constant shaking at 300 rpm for 2 hours and at room temperature. Additionally, the animal blood was spiked with known concentrations of the heavy metals or minerals of interest and a similar extraction procedure was used. FIG. 11 shows the results of subtraction between spiked blood samples and blank blood samples. The influence was compared in terms of how much the recoveries reported may be affected by having to subtract the background influences. This will increase the errors associated with the analysis.

The percentage of Mg, K, Ca, As, and Hg recovered from sheep blood are shown in Table 1.

TABLE 1
% Recovery of selected metals in sheep blood

| | Methacrylate | | | | Divinylbenzene | |
|---|---|---|---|---|---|---|
| | polymer PPM | | PKI 226 | | polymer PPM | |
| | Acid | Base | Acid | Base | Acid | Base |
| | extrac- | extrac- | extrac- | extrac- | extrac- | extrac- |
| | tion | tion | tion | tion | tion | tion |
| Mg | 139% | 120% | 220% | 237% | 136% | 128% |
| K | 102% | 65% | 116% | 80% | 104% | 69% |
| Ca | 192% | 71% | 239% | 231% | 152% | 67% |
| As | 169% | 486% | 233% | 791% | 351% | 551% |
| Hg | 18% | 96% | 11% | 163% | 9% | 65% |

Using Mg as an example, the data in Table 1 shows that 120-139% of the available Mg is extracted using two different PPM substrates of the present disclosure. These values are taken to be within acceptable error ranges for micro fluid samples. In contrast, over 220% of available Mg was extracted using a commercially available PKI 226 DBS substrate. This indicates that fluid samples taken and extracted with the PM 226 substrate contain Mg contaminants from an external source. As such, PPM substrates of the present disclosure would be expected to provide more reliable results for the analysis of Mg in blood.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgment of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications, and substitutions without departing from the scope of the invention as set forth and defined by the following claims.

1. A sampling device comprising a porous polymer monolith sampling substrate housed within a substantially impermeable housing, said housing surrounding the sampling substrate and further comprising a sampling aperture via which the sampling substrate is accessible externally from the sampling device.

2. The sampling device of claim 1, wherein the sampling aperture comprises a sampling capillary tube.

3-4. (canceled)

5. The sampling device of claim 1, wherein the sampling substrate comprises less than about 1 μg/cm² of contaminants.

6. (canceled)

7. The sampling device of claim 1, wherein the sampling substrate comprises porous polymeric divinylbenzene (DVB) or porous polymeric methacrylate.

8. The sampling device of claim 1, further comprising a hydrophilic coating on the porous polymer.

9. The sampling device of claim 8, wherein the hydrophilic coating is selected from one or more of the group consisting of polyethylene glycol (PEG), polyvinyl alcohol (PVA), polyvinylpyrrolidone (PVP), polyacrylic acid (PAA), polyacrylic maleic acid (PAMA), and poly(ethylene glycol)methyl ether methacrylate (PEGMA).

10. The sampling device of claim 9, wherein the hydrophilic coating comprises poly(ethylene glycol)methyl ether methacrylate (PEGMA).

11. (canceled)

12. The sampling device of claim 1, wherein the sampling substrate is coated with an anti-oxidant.

13. The sampling device of claim 12, wherein the anti-oxidant is selected from one or more of the group consisting of resveratrol, t-butylhydroquinone, BHT, BHA, citric acid, citrate, ascorbic acid, ascorbate, flavanoids, and antioxidant plant extracts.

14. The sampling device of claim 1, further comprising a removable seal or cap covering the sampling aperture.

15. An improved method of collecting and/or storing a fluid sample for future analysis that minimises contamination of the fluid sample, the method comprising:
providing the sampling device of claim 1;
collecting a fluid sample by contacting the sampling aperture directly or indirectly with a fluid under conditions for some of the fluid to transfer into the sampling substrate only through the sampling aperture; and
storing the sampling device with the sample sorbed into the sampling substrate for future analysis.

16. The method of claim 15, wherein the fluid sample to be collected and stored is a biological sample.

17. The method of claim 16, wherein the biological sample is a bodily fluid.

18. The method of claim 17, wherein the biological sample is blood.

19. The method of claim 17, wherein the bodily fluid contains or is suspected to contain a biomolecule of interest.

20. The method of claim 15, wherein the fluid sample to be collected and stored is an oil comprising fatty acids.

21. An improved method of collecting and/or storing a blood or blood plasma sample for future analysis that minimises contamination of the sample, the method comprising:
providing the sampling device of claim 1;
providing a blood sample;
collecting a blood sample of the blood by contacting the sampling aperture directly or indirectly with the blood under conditions for some of the blood to transfer into the sampling substrate only through the sampling aperture;
at least partially drying the blood sample on the sampling substrate; and
storing the sampling device with the blood sample absorbed into the sampling substrate for future analysis.

22. An improved method of collecting and/or storing a fluid sample for future analysis for the presence and/or amount of one or more metals, metal ions or essential minerals that minimises contamination of the sample, the method comprising:
providing the sampling device of claim 1;
providing a fluid to be analysed for one or more metals, metal ions or essential minerals;
collecting a fluid sample by contacting the sampling aperture directly or indirectly with the fluid under conditions for some of the fluid to transfer into the sampling substrate only through the sampling aperture;
optionally, at least partially drying the fluid sample on the sampling substrate;
storing the sampling device with the fluid sample sorbed into the sampling substrate for future analysis; and
determining the metal, metal ion or essential mineral composition of the fluid sample sorbed to the sampling substrate.

23. A kit for collecting and storing a blood sample from a subject, the kit comprising:
the sampling device of claim 1;
a sharp object for obtaining a blood sample from the subject; and
instructions for use.
